(19) United States
(12) Patent Application Publication
(10) Pub. No.: US 2004/0093644 A1
(43) Pub. Date: May 13, 2004

(54) Recombinant subunit proteins from porcine parvovirus produced in plants

(76) Inventors: Robert Rymerson, London (CA); Jim Brandie, London (CA); Lorne Babluk, Saskatoon (CA)

Correspondence Address: MORGAN LEWIS & BOCKIUS LLP, 1111 PENNSYLVANIA AVENUE NW, WASHINGTON, DC, 20004, US

(21) Appl. No.: 10/297,331

(22) PCT Filed: Jun. 5, 2001
(86) PCT No.: PCT/CA01/00818

(51) Int. Cl. A01H 1/00; C12N 15/82; A61K 35/78

(52) U.S. Cl. 800/288; 424/751

(57) ABSTRACT

The present invention discloses a method for producing a protein in a plant comprising i) transforming the plant with a nucleotide sequence which expresses a porcine parvovirus VP2 subunit protein, or a fragment or a derivative thereof, and ii) growing the transformed plant. The present invention also teaches harvesting and feeding the transgenic plant, or an extract obtained from the transgenic plant to animals. Further, the protein produced by the method of the present invention may be partially or fully purified from the plant, and optionally reformulated into an alternate dosage form and administered to an animal. The protein may be used to confer resistance to porcine parvoviuus in an animal.

The present invention relates to the production of recombinant viral proteins in plants. More specifically, the invention relates to expression of porcine parvovirus proteins in plants. Still more specifically, the present invention relates to expression of porcine parvovirus VP2 subunit proteins in plants.

### BACKGROUND OF THE INVENTION

Porcine parvovirus is an autonomous replicating virus containing a single stranded DNA molecule of about 5100 nucleotides (Molitor et al., 1984). Only the minus strand of the DNA is packaged into virions. The genome of the virus encodes three capsid proteins (VP1, VP2, VP3) and one non-structural protein (NS1). The capsid of parvovirus is about 22-25 nanometers in diameter and is comprised of VP1 and VP2 subunits. These proteins are derived from alternatively spliced versions of the same RNA molecule and thus overlap in sequence. Further, porcine parvovirus exhibits a high level of sequence similarity to feline panleukopenia virus, canine parvoviruses and rodent parvovirus (Ranz et al., 1989). Porcine parvovirus has been linked to fetal death and mummification, reproductive failure in sows, diarrhea in young piglets, and a delayed return to oestrus (Dunne et al 1965; Joo and Johnson, 1976; Mengeling, 1978). Also, certain isolates or strains of the virus may cause skin lesions in pigs.

Serological studies show that porcine parvovirus is widespread in all swine-producing regions of the world with up to 80% of animals showing seroconversion. Although there are differences in porcine parvovirus strains, some being extremely pathogenic and others being less pathogenic or totally non-pathogenic, when the virus becomes established or endemic in a country, the pathogenic strains appear to circulate in the population.

VP2 proteins are known to self-assemble into virus-like particles (VLPs) when produced in a baculovirus expression system (Martinez et al., 1992). The VLPs have high immunogenic activity and are able to elicit protective immunity when administered systemically to pigs (Martinez et al., 1992). However, the production of protein in baculovirus expression systems is complex and must be performed in a laboratory or similar setting. Further, the protein must be purified from baculovirus. The purification of protein from baculovirus increases the cost of protein production.

Molecular farming offers the potential for low-cost production of large amounts of protein that closely resembles that of the original host organism in both conformation and biological activity. Several proteins have been produced in plants including γ-interferon in tobacco (U.S. Pat No. 5,625,175), norwalk virus capsid protein in transgenic tobacco and potato plants (Mason et al. 1996), B subunit toxin of Vibrio cholerae in potato plants (Arakawa et al. 1998), and antigenic peptides from both rabies virus and mink enteritis virus in alfalfa.

Expressing transgenic proteins in plants offers many advantages over expressing transgenic proteins in other organisms such as bacteria. First, plants are higher eukaryotic organisms and thus have the same or similar intracellular machinery and mechanisms which govern protein folding, assembly and glycosylation as do mammalian systems. Further, unlike fermentation-based bacterial and mammalian cell systems, protein production in plants is not restricted by physical facilities. For example, agricultural scale production of recombinant proteins by plants is likely to be significantly greater than that produced by fermentation-based bacterial and mammalian cell systems. In addition, the costs of producing recombinant proteins in plants may be 10 to 50-fold lower than conventional bacterial bioreactor systems (Kusnadi et al. 1997). Also, plant systems produce pathogen free recombinant proteins. Further, the ability to produce biologically-active recombinant proteins in edible plant tissues or extracts allows low-cost oral delivery of proteins such as antigens as feed additives, and potentially eliminates the need for expensive down-stream purification processes of the protein.

There is a need in the art for proteins which confer protection against porcine parvovirus and the diseases caused by porcine parvovirus. Further, there is a need in the art for proteins which confer protection against porcine parvovirus and the diseases caused by porcine parvovirus and which may be produced in large quantities in plants. Also, there is a need for transgenic plants that express an immuno-reactive antigen of the porcine parvovirus which may be produced in relatively large quantities and purified.

It is an object of the present invention to overcome drawbacks in the prior art.

The above object is met by a combination of the features of the main claims. The sub-claims disclose further advantageous embodiments of the invention.

### SUMMARY OF THE INVENTION

The present invention relates to the production of recombinant viral proteins in plants. More specifically, the invention relates to expression of porcine parvovirus proteins in plants. Still more specifically, the present invention relates to expression of porcine parvovirus VP2 subunit proteins in plants.

According to the present invention there is provided a method for producing a protein in a plant, comprising:

i) transforming the plant with a nucleotide sequence expresses a porcine parvovirus VP2 subunit protein, or a fragment or a derivative thereof; and

ii) growing the transformed plant.

The present invention also relates to a plant, plant seed, plant cell, or progeny produced therefrom comprising a nucleotide sequence that encodes a porcine parvovirus VP2 subunit protein, or a fragment or a derivative thereof, produced by the above method.

The present invention also relates to a method as described above which further comprises harvesting the plant and administering the plant to an animal. Alternatively, the plant may be harvested, and the protein purified. Optionally, the purified protein may be formulated into a dosage form and administered to an animal.

The present invention also relates to a porcine parvovirus VP2 subunit protein, a fragment or derivative thereof produced by the above method.

The present invention also relates to protein produced according to the method of the present invention that is fed to pigs to confer resistance to porcine parvovirus.

The present invention also relates to protein produced according to the method of the present invention that is administered to pigs and that confers resistance to porcine parvovirus at a mucosal membrane of the animal.

This summary does not necessarily describe all necessary features of the invention but that the invention may also reside in a sub-combination of the described features.

### BRIEF DESCRIPTION OF THE DRAWINGS

These and other features of the invention will become more apparent from the following description in which reference is made to the appended drawings wherein:

FIG. 1 shows the general structure of a binary transformation vector (pCAVP) useful for the introduction of a AMV-VP2 gene into plants.

FIG. 2 shows the DNA coding sequence of the AMV-VP2 gene (SEQ ID NO:1).

FIG. 3 illustrates detection of the VP2 coding region in plants by PCR using untransformed control DNA (81V9), pCAVP plasmid DNA (C) and putative transgenic plant DNA (1-13).

FIG. 4 shows Reverse transcriptase PCR (RT-PCR) analysis of transgeneic plants containing a AMV-VP2 transgene. Products of RT-PCR reactions were resolved on a 1% agarose gel, and the VP2-specific product was approximately 1 KB. The numbers indicate the VP2-containing plant, with “+” and “−” indicating the addition or omission of reverse transcriptase in the first strand reaction. “C” contained RNA isolated from an untransformed plant. “P” stands for the plasmid positive control for the PCR. “W” contained water in place of template.

FIG. 5 shows a transmission electronic micrograph of a VP2-expressing tobacco plant. Virus-like particles of approximately 25 nm in diameter that accumulate in the cytoplasm are indicated with an arrow. The bar represents 50 nm.

FIG. 6 shows western analysis of transgenic plants carrying a AMV-VP2 gene (lanes 1,2, 5, 7, 9, 11,13,and 14) and an untransformed control plant (81V9).

FIG. 7 shows the results of a serum neutralization assay using fetal swine testicle cells, porcine parvovirus, and serum from immunized mice. The mice in groups VP2-9-1 and VP-9-2 were immunized with plant extract from a VP2-expressing plant, while groups 81V9-1, 81V9-2 and 81V9-3 received plant extract from untransformed control plants.

### DESCRIPTION OF PREFERRED EMBODIMENT

The present invention relates to the production of recombinant viral proteins in plants. More specifically, the invention relates to expression of porcine parvovirus proteins in plants. Still more specifically, the present invention relates to expression of porcine parvovirus VP2 subunit proteins in plants.

According to an aspect of an embodiment of the present invention, there is provided a method of producing a protein in a plant comprising i) transforming the plant with a nucleotide sequence which expresses a porcine parvovirus VP-2 protein, a fragment, or derivative thereof, and ii) growing the transformed plant. In another aspect of an embodiment, the transformed plant may be harvested and fed to an animal. In still another aspect of an embodiment the transformed plant may be harvested, the protein partially or fully purified and then optionally formulated into a dosage form and administered to an animal.

The porcine parvovirus VP-2 protein, fragment, or derivative thereof, produced by the method of the present invention may administered to an animal to confer resistance to porcine parvovirus.

The present invention also relates to methods as described above wherein the porcine parvovirus VP-2 protein, a fragment, or derivative thereof is partially or fully purified, reformulated into a dosage form and used as a vaccine in an animal.

The present invention also relates to a protein produced by the method described above that induces a mucosal immune response that renders an animal immune to virus penetration at a mucosal site.

The porcine parvovirus VP-2 protein, fragment, or derivative thereof may be recognized as foreign by the immune system of an animal and may be capable of eliciting an immune response in the animal such that the animal acquires immunity to subsequent challenges by a virus, bacterium, or agent which comprises the protein or fragment or derivative thereof. In an embodiment of the present invention, the protein, fragment or derivative thereof is the porcine parvovirus VP2 subunit protein, a fragment of porcine parvovirus VP2 subunit protein, or a derivative of porcine parvovirus VP2 subunit protein.

A “derivative” includes any substitution, deletion, or addition to the nucleotide or amino acid sequence of the porcine parvovirus VP2 subunit protein, for example, but not limited to, the AMV-VP2 chimeric construct, provided that the derivative thereof, maintains the property of conferring resistance to a porcine parvovirus infection. A “fragment” includes any derivative of the porcine parvovirus VP2 subunit protein where one or more amino acid residues have been deleted from the amino terminus, the carboxy terminus or both the amino terminus and the carboxy terminus of the full length protein, provided that the fragment retains the capacity of conferring resistance to a porcine parvovirus infection.

By the term ‘conferring resistance’ it is meant stimulation of the immune system of an animal in response to foreign matter, such as but not limited to a protein, such that the foreign matter is neutralized and degraded by the immune system of an animal during a subsequent challenge. Stimulation of the immune system may include but is not limited to an increase in the production of a specific antibody against an antigen or a general increase in the production of many antibodies, increased production of immune cells, such as but not limited to macrophages and lymphocytes, or increased production of immunomodulators such as but not limited to interferons and interleukins, or a combination thereof. In addition the immune response may occur at a specific site, for example, but not wishing to be limiting, a mucousal membrane which may be a site of entry of foreign matter.

The present invention provides an effective method for the reliable production of VP2 or a fragment or derivative thereof. In order to optimize the expression of the foreign gene within plants, the native or synthetic gene may be used or altered as required so that the corresponding protein is produced at a level higher than the native gene. For example, which is not to be considered limiting, the gene may be a synthetic gene, optimized for codon usage within a plant, comprising at least about 80% homology with the native VP2 as determined using sequence comparison techniques for example but not limited to BLAST algorithm (GenBank: www.ncbi.nlm.nih.gov/cgi-bin/BLAST/), using default parameters (Program: blastn; Database: nr; Expect 10; filter: low complexity; Alignment: pairwise; Word size: 11). Analogs, or derivatives thereof, also include those DNA sequences which hybridize under stringent hybridization conditions (see Maniatis et al., in Molecular Cloning (A Laboratory Manual), Cold Spring Harbor Laboratory, 1982, p. 387-389) to the DNA sequence of SEQ ID NO: 1 provided that the sequences exhibit the property as described herein. It is also contemplated that fragments or portions of VP2 or derivatives thereof, that exhibit useful biological properties, for example, but not limited to antigenic properties, may be expressed within plant tissues. Preferably, fragments or portions of VP2, or derivatives thereof, exhibit properties that aid in the health and productivity of animals similar to those observed with the administration of native VP2.

Furthermore, other modifications of the native VP2 or synthetic VP2 (sVP2) gene may occur so that expression of the gene, and stability or purification of the protein can be optimized. For example, modification of the 5′ or 3′ region of these genes can be carried out in order to enhance expression of the gene and target the product to an appropriate intercellular compartment to ensure stability. An example of 5′modification may include a signal peptide (signal sequence) to direct the protein to a specific cellular compartment, for example which is not to be considered limiting in any manner, the signal sequence from a tobacco pathogenesis related (PR) protein (Cornelissen et al. 1986, Nature 321:531-532), or the alfalfa mosaic virus (AMV) leader sequence (Jobling and Gehrke, 1987, Nature 325:622-625). An example of a 3′ modification includes a histidine tag which can be used to aid in purification of the encoded protein. However, other alterations to the 5′, 3′, regions, or internal modifications may be utilized in order to optimize expression, stability and, optionally, purification of the expressed protein. It is preferred that the synthetic gene sVP2, encoding the mature protein comprises a codon bias similar to highly expressed genes found in plants carry, and if desired, comprises a motif that allows for simple extraction, for example an affinity tag, such as but not limited to a His-tag as is known in the art. The affinity-tag of the protein may be used for the purification of the protein using chromatography, for example a Ni²⁺ column may be used for the purification of HIS-tag containing proteins. It is also contemplated that the protein may be modified so that the immature protein is targeted to a compartment of the cell to enhance stability of the product, for example the plastid, or the lumen of the endoplasmic reticulum (ER). However, other sites may also be targeted for example, extracellular secretion, in order to simplify extraction protocols, or the mitochondria compartment.

By the term “expression” is meant the production of a functional RNA, protein or both, from a gene or transgene.

By “gene”, it is meant a particular sequence of nucleotides including the coding region, or fragment thereof, and optionally the promoter and terminator regions which regulates expression of the gene, as well as other sites required for gene expression for example a polyadenylation signal which regulates the termination of transcription. By “coding region” or “structural gene”, it is meant any region of DNA that determines the primary structure of a polypeptide following genetic transcription and translation. Furthermore, fragments comprising regions of interest of a coding region or structural gene may also be employed as needed. By “transgene” is meant genes that have been introduced into a host cell or organism that would not normally be present in the host cell or organism, particularly nucleic acids that have been modified by recombinant DNA techniques. The term “transgene” also includes host genes that are placed under the control of a new promoter or terminator sequence, for example. By “synthetic gene” it is meant a DNA sequence of a structural gene that is synthesized using methods known in the art for example but not limited to chemical syntheses, site directed mutagenesis, or PCR and related techniques. A synthetic gene can comprise a fragment or the entire coding region of VP2. Furthermore, a synthetic gene may also comprise regulatory elements that enhance expression of the gene, or motifs that aid in the stability or cellular targeting of the protein product. It is also contemplated that a synthetic gene optionally includes regions useful for the isolation and purification of the protein, or the protein fragment, encoded by the synthetic gene such as an affinity-tag.

By “codon optimization” it is meant the selection of appropriate DNA nucleotides for the synthesis of oligonucleotide building blocks, and their subsequent enzymatic assembly, of a structural gene or fragment thereof in order to approach codon usage within plants.

In order to maximize expression levels and transgene protein production of VP2 or fragment or derivatives or fragments thereof, the nucleic acid sequence of VP2 is examined and the coding region modified to optimize for expression of the gene in plants, using a procedure similar to that outlined by Sardana et al. (Plant Cell Reports 15:677-681; 1996). A table of codon usage from highly expressed genes of dicotyledonous plants is compiled using the data of Murray et al. (Nuc Acids Res. 17:477-498; 1989). This information is used to compile a synthetic sequence for sVP2.

Assembly of the sVP2 gene of this invention is performed using standard technology know in the art. The gene may be assembled enzymatically, within a DNA vector, for example using PCR, or synthesised from chemically synthesized oligonucleotide duplex segments. The synthetic VP2 gene is then transformed to plant genomes using methods known in the art. Expression of the gene may be determined using methods known within the art, for example Northern analysis, or ELISA.

Therefore, the present invention is directed to a gene for VP2 and optimizing its expression in plants. The sVP2 is synthesized to contain a suitable signal sequence, for example the AMV leader sequence, and a 6× histidine tail.

In an aspect of an embodiment, the method of the present invention relates to transforming a plant with a chimeric construct which expresses a porcine parvovirus VP-2 protein, a fragment, or a derivative thereof in a plant. The chimeric construct comprises a nucleotide sequence which encodes a porcine parvovirus VP2 protein, a fragment or a derivative thereof. The chimeric construct further comprises regulatory sequences in operative association with the gene encoding a porcine parvovirus VP2 protein, a fragment or a derivative thereof. Any regulatory sequence may be used, for example, but not limited to a constitutive sequence.

A constitutive sequence directs the expression of a gene throughout the various parts of a plant and continuously throughout plant development. Examples of known constitutive sequences include promoters associated with the CaMV 35S transcript. (Odell et al., 1985, Nature, 313: 810-812), the rice actin 1 (Zhang et al, 1991, Plant Cell, 3: 1155-1165) and triosephosphate isomerase 1 (Xu et al, 1994, Plant Physiol. 106: 459-467) genes, the maize ubiquitin 1 gene (Cornejo et al, 1993, Plant Mol. Biol. 29: 637-646), the Arabidopsis ubiquitin 1 and 6 genes (Holtorf et al, 1995, Plant Mol. Biol. 29: 637-646), and the tobacco translational initiation factor 4A gene (Mandel et al, 1995 Plant Mol. Biol. 29: 995-1004). The term “constitutive” as used herein does not necessarily indicate that a gene under control of the constitutive sequence is expressed at the same level in all cell types, but that the gene is expressed in a wide range of cell types even though variation in abundance is often observed.

A regulatory sequence may also include, but is not limited to promoter elements, basal (core) promoter elements, elements that are inducible in response to an external stimulus, elements that mediate promoter activity such as negative regulatory sequences or transcriptional enhancers. Regulatory sequences may also comprise elements that are active following transcription, for example, regulatory sequences that modulate gene expression such as translational and transcriptional enhancers, translational and transcriptional repressors, upstream activating sequences, and mRNA instability determinants. Several of these latter elements may be located proximal to the coding region. In the context of this disclosure, the regulatory sequence typically refers to a sequence of DNA, usually, but not always, upstream (5′) to the coding sequence of a structural gene, which controls the expression of the coding region by providing the recognition for RNA polymerase and/or other factors required for transcription to start at a particular site. However, it is to be understood that other nucleotide sequences, located within introns, or 3′ of the sequence may also contribute to the regulation of expression of a coding region of interest. An example of a regulatory sequence that provides for the recognition for RNA polymerase or other transcriptional factors to ensure initiation at a particular site is a promoter sequence. A promoter sequence comprises a basal promoter sequence, responsible for the initiation of transcription, as well as other regulatory sequences (as listed above) that modify gene expression.

There are also several types of regulatory sequences, including those that are developmentally regulated, inducible and constitutive. A regulatory sequence that is developmentally regulated, or controls the differential expression of a gene under its control, is activated within certain organs or tissues of an organ at specific times during the development of that organ or tissue. However, some regulatory sequences that are developmentally regulated may preferentially be active within certain organs or tissues at specific developmental stages, they may also be active in a developmentally regulated manner, or at a basal level in other organs or tissues within the plant as well.

An inducible regulatory sequence is one that is capable of directly or indirectly activating transcription of one or more DNA sequences or genes in response to an inducer. In the absence of an inducer the DNA sequences or genes will not be transcribed. Typically the protein factor, that binds specifically to an inducible sequence to activate transcription, may be present in an inactive form which is then directly or indirectly converted to the active form by the inducer. However, the protein factor may also be absent. The inducer can be a chemical agent such as a protein, metabolite, growth regulator, herbicide or phenolic compound or a physiological stress imposed directly by heat, cold, salt, or toxic elements or indirectly through the action of a pathogen or disease agent such as a virus. A plant cell containing an inducible sequence may be exposed to an inducer by externally applying the inducer to the cell or plant such as by spraying, watering, heating or similar methods. Inducible elements may be derived from either plant or non-plant genes (e.g. Gatz, C. and Lenk, I. R. P.,1998, Trends Plant Sci. 3, 352-358; which is incorporated by reference). Examples, of potential inducible promoters include, but not limited to, teracycline-inducible promoter (Gatz, C.,1997, Ann. Rev. Plant Physiol. Plant Mol. Biol. 48, 89-108; which is incorporated by reference), steroid inducible promoter (Aoyama, T. and Chua, N. H.,1997, Plant J. 2, 397-404; which is incorporated by reference) and ethanol-inducible promoter (Salter, M. G., et al, 1998, Plant Journal 16, 127-132; Caddick, M. X., et al,1998, Nature Biotech. 16, 177-180, which are incorporated by reference) cytokinin inducible IB6 and CK11 genes (Brandstatter, I. and Kieber, J. J.,1998, Plant Cell 10, 1009-1019; Kakimoto, T., 1996, Science 274, 982-985; which are incorporated by reference) and the auxin inducible element, DR5 (Ulmasov, T., et al., 1997, Plant Cell 9, 1963-1971; which is incorporated by reference).

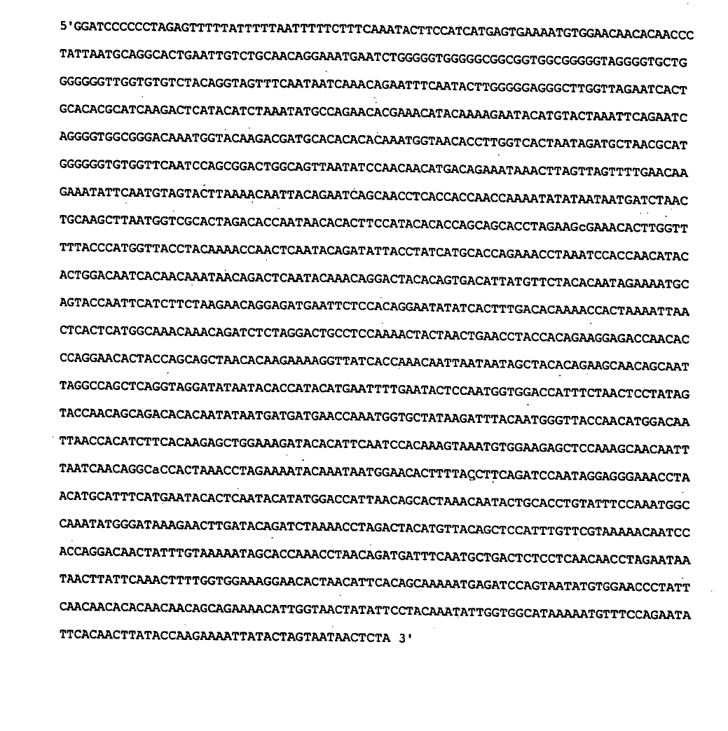

The nucleotide sequence of the method of the present invention thus includes the DNA sequences of SEQ ID NO: 1 (see also FIG. 2), and fragments or derivative thereof, as well as analogues of, or nucleic acid sequences comprising about 80% similarity with the nucleic acids as defined in SEQ ID NO: 1. Analogues include those DNA sequences which hybridize under stringent hybridization conditions (see Maniatis et al., in Molecular Cloning (A Laboratory Manual), Cold Spring Harbor Laboratory, 1982, p.387-389) to the DNA sequence of SEQ ID NO: 1, provided that said sequences maintain at least one property of the activity of the gene as defined herein.

The VP2 of the present invention may be introduced into any desired plant, including forage plants, food crops, or other plants depending upon the need. Examples of such plants include, but not limited to, alfalfa, corn, barley, tobacco, and potato.

According to another aspect of an embodiment of the present invention, the protein produced by the method of the present invention may be administered to an animal, such as but not limited to a pig, in the form of a dietary supplement. In such an embodiment the plant producing the protein may be added directly to the chow that is consumed by the pig. If the protein is orally administered, the plant tissue may be harvested and directly fed to the animal, or the harvested tissue may be dried prior to feeding, or the animal may be permitted to graze on the plant with no prior harvest taking place. It is also considered within the scope of this invention for the harvested plant tissues to be provided as a food supplement within animal feed. If the plant tissue is being fed to an animal with little or no further processing-it is preferred that the plant tissue being administered is edible.

Alternatively, the protein produced by the method of the present invention may be partially or completely purified from the plant and reformulated into a desired dosage form. The dosage form may comprise, but is not limited to an oral dosage form wherein the protein is dissolved in a suitable excipient such as but not limited to water. In addition, the protein may be formulated into an injectable dosage form. An injectable dosage form may include other adjuvants that may function to enhance the immunogenicity of the protein. For example, but not meant to be limiting, the protein may be combined with Freund's adjuvant. Also, the protein produced by the method of the present invention may be formulated into a vaccine or used in the production of a medicament. In this latter case, the protein may be produced in either edible or non-edible plants.

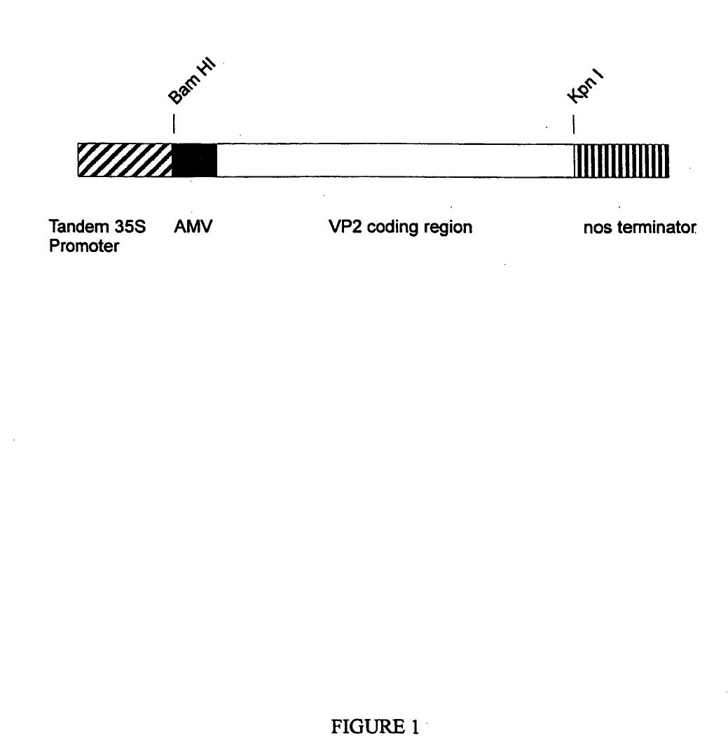

In an embodiment of the method of the present invention, the coding region of the VP2 subunit protein may be linked to as part, for example, but not limited to the alfalfa mosaic virus leader sequence and the fused sequence may be cloned into a vector suitable for expression in a plant, for example, but not limited to a pCAVP binary vector (FIG. 1) comprising a desired regulatory region, for example, but not limited to a tandem 35S CaMV promoter and a nos terminator. The pCAVP binary vector comprising the cloned AMV-VP2 gene may be introduced into a transforming strain, for example, but not limited to an Agrobacterium tumefaciens strain containing a disarmed Ti plasmid, and plants may be transformed using methods described in the art. However, as one of skill in the art will understand, there exists many other vectors, promoters, terminators and transformation systems which may be used in place of those described herein. For example, but not wishing to be limiting, the constructs of the present invention can be introduced into plant cells using Ti plasmids, Ri plasmids, plant virus vectors, direct DNA transformation, micro-injection, electroporation, etc. For reviews of such techniques see for example Weissbach and Weissbach, Methods for Plant Molecular Biology, Academy Press, New York VIII, pp. 421-463 (1988); Geierson and Corey, Plant Molecular Biology, 2d Ed. (1988); and Miki and Iyer, Fundamentals of Gene Transfer in Plants. In Plant Metabolism, 2d Ed. D T. Dennis, D H Turpin, D D Lefebrve, D B Layzell (eds), Addison Wesly, Langmans Ltd. London, pp. 561-579 (1997). The present invention further includes a suitable vector comprising the chimeric gene construct.

To aid in identification of transformed plant cells, the constructs of this invention may be further manipulated to include plant selectable markers. Useful selectable markers include enzymes which provide for resistance to chemicals such as an antibiotic for example, gentamycin, hygromycin, kanamycin, or herbicides such as phosphinothrycin, glyphosate, chlorosulfuron, and the like. Similarly, enzymes providing for production of a compound identifiable by colour change such as GUS (β-glucuronidase), or luminescence, such as luciferase or GFP, are useful.

The method of the present invention may be practiced in any plant. For example, but not wishing to be limiting, the present invention may be practiced in tobacco, corn, barley, alfalfa and potato. Preferably, the method of the present invention is practiced in low nicotine tobacco plants.

In an aspect of an embodiment, the method of the present invention relates to transforming a plant with a chimeric construct which expresses a porcine parvovirus VP-2 protein, a fragment, or a derivative thereof in a plant.

The chimeric construct comprises a nucleotide sequence which encodes a porcine parvovirus VP2 protein, a fragment or a derivative thereof. Therefore the present invention includes plants, plant cells or plant seeds comprising a nucleotide sequence which encodes a porcine parvovirus VP2 protein, a fragment or a derivative thereof.

The protein produced by the method of the present invention may comprise full-length porcine parvovirus VP2 subunit protein or a fragment or derivative thereof. As will be appreciated by someone of skill in the art, an entire protein may not be required to immunize an animal to subsequent challenges by the protein or an infectious agent comprising the protein, but rather, it may be possible that a smaller fragment of the protein can immunize an animal against subsequent challenges by the full-length protein, or an infectious agent comprising the full-length protein, such as but not limited to a bacterium or a virus. Further, as someone of skill in the art will understand, viruses often undergo mutations in genes which permit the virus to escape immune detection in an animal. Thus, the method of the present invention also contemplates that different porcine parvovirus strains may have different VP2 subunit protein sequences and therefore natural variants or man-made variants of the porcine parvovirus VP2 subunit protein, fragment or derivative thereof are fully contemplated to be included by the present invention.

The protein produced by the method of the present invention may be partially or completely purified from the plant. In addition, the protein may be formulated into an injectable dosage form. An injectable dosage form may include other adjuvants that may function to enhance the immunogenicity of the protein. For example, but not meant to be limiting, the protein or partially purified protein may be combined with Freund's adjuvant. Thus, the protein produced by the method of the present invention confers immunity against parvovirus in animals that have not been previously challenged with the virus, when the same animals are injected with a dosage form of a protein composition comprising the partially or completely purified protein produced by the method of the present invention. For example, but not meant to be limiting, when mice are injected with a dosage form of a plant extract comprising the protein produced by the method of the present invention an immune response is elicited, such that serum collected from the injected mice is capable of conferring resistance against porcine parvovirus in an in vitro assay. Furthermore, the protein produced by the method of the present invention may be used to confer immunity to parvovirus in animals that have not been previously challenged with the virus, by feeding the same animals transformed plants expressing the protein. Similar immunity may be conferred when animals are fed an oral solution comprising the protein produced by the method of the present invention that was partially or completely purified. In addition, all of the above results are observed regardless of whether the protein produced by the method of the present invention contained full-length VP2 subunit protein, a fragment or a derivative thereof.

The protein produced by the method of the present invention may be used to confer immunity to porcine parvovirus in pigs that have not been previously challenged with the virus, by feeding pigs transformed plants expressing the protein. Similar immunity may be conferred when pigs are fed an oral solution comprising the protein produced by the method of the present invention that was partially purified or fully purified. The protein produced by the method of the present invention also confers immunity against porcine parvovirus in pigs that have not been previously challenged with the virus, when the same pigs were injected with a dosage form of the protein that was partially purified or fully purified. Furthermore, the protein produced by the method of the present invention may be used to induce mucosal immunity against porcine parvovirus in pigs that had not been previously challenged with the virus, when the same pigs were fed with protein containing plant material or injected with a dosage form of the protein that was partially purified or fully purified. In addition, all of the above results are observed regardless of whether the protein produced by the method of the present invention contained full-length VP2 subunit protein, a fragment or a derivative thereof.

The protein produced by the method of the present invention may induce immunity at mucus membranes such as but not limited to nasal mucus membranes and gastrointestinal mucus membranes. Inducing immunity at mucus membranes may permit neutralization of the virus at the site of entry of the animal. Further, serum antibody isotopes may be assayed to determine whether killed VLPs can stimulate immune cells, such as but not limited to macrophages, helper T-cells, cytotoxic T-cells, neutrophils, or combinations thereof. In addition, the presence of cytokines such as but not limited to interferons, and interleukins may be assessed. Results suggest that there may be a correlation between the levels of cytokines, the levels of immune cells or the presence of serum antibodies in animals treated with the protein produced by the method of the present invention.

Following immunization, pregnant sows may be challenged, without wishing to be limiting, orally, and the level of protection may be determined. The level of protection may be assessed by monitoring clinical signs and assessing the ability of the virus to cross the placenta and infect the fetus. In the fetus, the presence of viral antigens can be assayed by immunohistochemical studies and by virus isolation.

The protein produced by the method of the present invention, which comprises viral recombinant sub-unit proteins and fragments thereof may have a variety of uses including, but not limited to the production of immunogenically active sub-unit proteins for use as oral proteins, the production of immunogenically active sub-unit proteins for the use as systemically administered proteins, the production of antibodies for veterinary diagnostic use, for general research purposes or combinations thereof. Further, the protein produced by the method of the present invention may be produced in large quantities in plants, isolated and optionally purified at potentially reduced costs compared to other conventional methods of producing proteins such as but not limited those which employ fermentation processes. It is also contemplated that the VP2 protein, fragments or derivatives thereof may be used to carry epitopes from other anitgens. Such a hybrid protein may also be used to elicit protective immunity to the animal.

The above description is not intended to limit the claimed invention in any manner, furthermore the discussed combination of features might not be absolutely necessary for the inventive solution.

The present invention will be further illustrated in the following examples. However, it is to be understood that these examples are for illustrative purposes only, and should not be used to limit the scope of the present invention in any manner.

### EXAMPLE 1

### Construction of VP2 Plant Expression Vectors

The alfalfa mosaic virus (AMV) leader sequence (Jobling and Gehrke, 1987) is added to the VP2 gene by PCR (FIG. 1). A 70 nucleotide primer (AMVVP; SEQ ID NO:2):

5′GGGTCTAGAGTTTTTATTTTTAATTTTTCTTTCAAA
TACTTCCATCATGAGTGAAAATCTGGAACAACAC-3′

is synthesized. PCR amplification is performed using 5.25 units of Expand DNA polymerase (Boehringer Mannheim), 350 μmol dNTPs, 1.75 mM MgCl₂, 10 pmole of AMVVP and 77 primers and 5 ng of template (pBluescript KS+ containing the VP2 gene). Thirty PCR cycles are performed according to the manufacturer's instructions, using an annealing temperature of 65° C. The amplified DNA fragment is purified using standard methods in the art, end filled, and introduced into the Sma I restriction site of pBluescript KS+. The resulting plasmid (pBSAVP) is digested with BamHI and KpnI restriction enzymes and the AMV-VP2 fragment, shown in FIG. 2, is introduced into the binary vector pCamter X which was previously digested with the same enzymes. This binary vector contains a duplicated 35S promoter (Kay et al 1987), a multiple cloning region, the nos terminator, and further comprises neomycin phosphotransferase II (npt II) as the selectable marker between the T-DNA border sequences. Similar results may be obtained using the tCUP promoter (Miki et al. 1998).

### EXAMPLE 2

### Plant Transformation with the VP2 Expression Vector

The pCAVP binary vector is introduced into Agrobacterium tumefaciens (EHA 105 which contains the disarmed Ti plasmid pEHA 104) using standard protocols within the art (Horsch et al. 1985). Low nicotine tobacco plants from the cultivar 81-V9 are grown aseptically for transformation. The midvein of the leaf material is excised and the leaf material is cut into 1 cm² fragments using a scalpel. The fragments are precultured with the epidermal side down for 2 days on solid media consisting of MS (Murishage and Skoog) salts, B5 vitamins, 3% sucrose, 1 mg/L 6-benzylaminopurine (BA) and 0.1 mg/L α-naphthalene acetic acid (NAA).

Infection of leaf explants with transgenic Agrobacterium from cultures grown overnight in LB (Luria Broth) medium is achieved by submerging leaf explants in the agrobacterium cultures that were diluted about 50%, followed by blotting on Whatman No.2 filter paper to remove excess bacteria. The explants are returned to the solid medium described above and allowed to grow for 2 days. Subsequently, bacterial growth was inhibited and selection for transformants was initiated by transferring explants to medium containing 500 μg/mL Timentin and 100 μg/mL kanamycin. The explants were transferred to fresh media every 2-3 weeks until shoots emerged.

After the explants develop well-defined stems, the shoots are excised and transferred to Magenta boxes containing solid media. The solid media comprises MS salts, B5 vitamins, 3% sucrose, 500 μg/ml Timentin and 100 μg/ml kanamycin. Roots are allowed to develop from the putative transgenic plants at which point the plants are placed in soil-less potting mix in a 15 cm plastic pot and transferred to the greenhouse. To ensure that each plant has arisen from an independent transformation event the explants are divided into separate fragments early in the procedure and only 1 shoot was selected from each fragment.

### EXAMPLE 3

### Analysis of the AMV-VP2 Insertions in Transgenic Plants

Each of the putative transgenic tobacco plants resulting from the agrobacterium transformation is screened for the presence of the VP2 gene using PCR. The primers chosen for PCR amplification are VP1060F:

| | |
|---|---|
| GGTGGACCATTTCTAACTC | (SEQ ID NO:3) |
| and nos: | |
| CCGGCAACAGGATTCAATCTTAA. | (SEQ ID NO:4) |

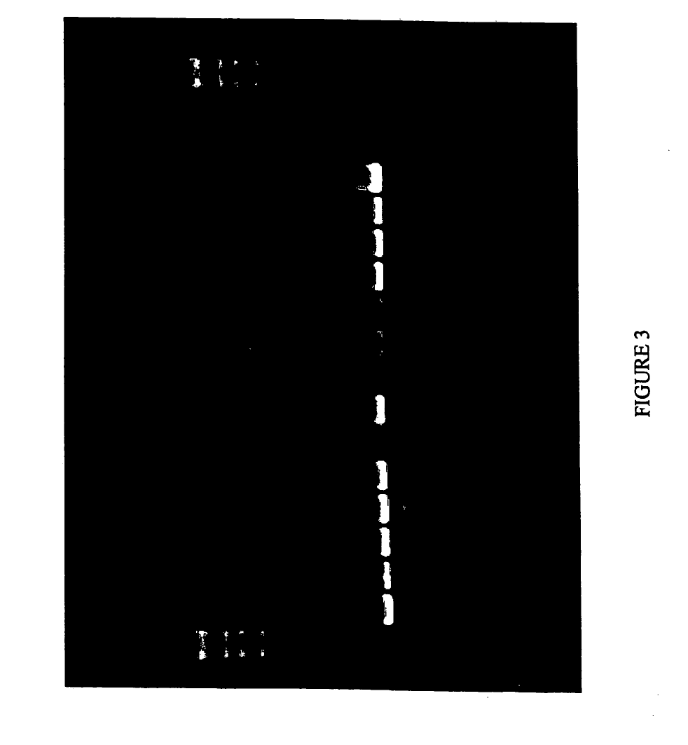

DNA is extracted from plant tissue using standard methods known in the art and is subjected to 35 cycles of amplification using 10 pMol of both primers, 100 mM dNTPs, 2mM MgCl₂, 1 U Taq polymerase and approximately 50 ng of plant DNA. Thirteen plants were confirmed positive by PCR, as shown in FIG. 3.

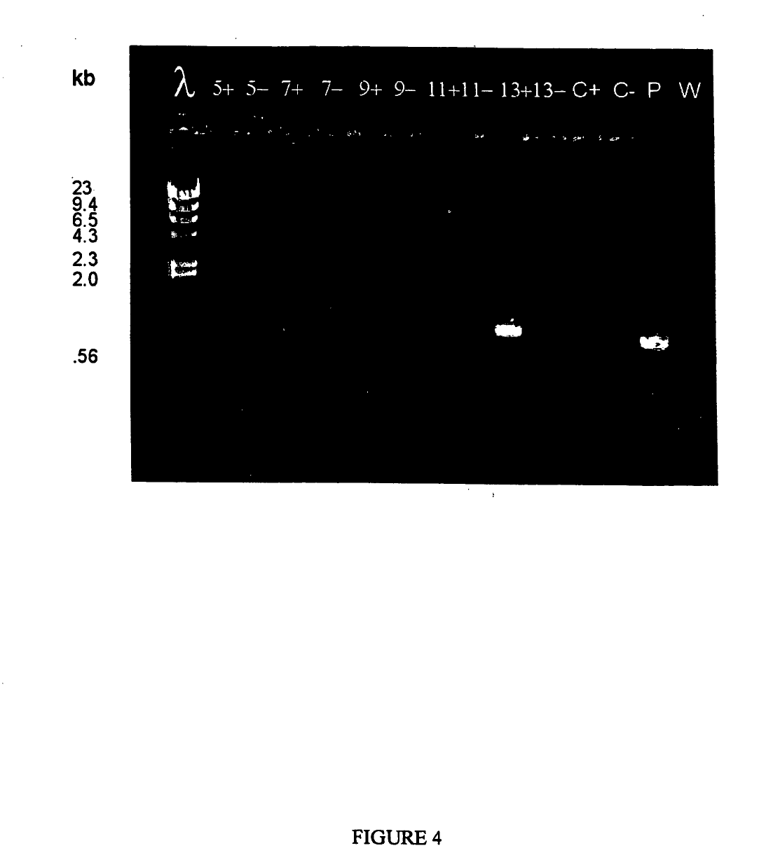

Transgenic tobacco plants are assayed for expression of VP2 RNA by RT-PCR For RT-PCR, 10 micrograms of RNA was treated with RNase-free DNase and 5 ug of this was used for cDNA synthesis. Oligo dT (12-18) (0.05 ug) was mixed with the RNA incubated at 70° C., and chilled on ice. First strand synthesis proceeded for 50 minutes at 42° C. in a buffer containing 10 mM DTT. 0.5 mM dNTP, 50 mM Tris HCl pH 8.3, 75 mM KCl, 3 mM MgCl₂, and 200 units of Superscript II reverse transcriptase (Gibco BRL Life Technologies). The enzyme was heat-inactivated at 70° C for 15 minutes. As a negative control, reverse transcription reactions that did not contain any reverse transcriptase were done for each sample. PCR was then done as described above with 2 uL of the reverse transcription reaction as the template and primers specific to VP2. The synthesis of the first strand was confirmed by testing it as a template for PCR with primers for the actin gene. Transcript for VP2 was detected in five of the plants tested as shown in FIG. 4.

### EXAMPLE 4

### Detection of the VP2 Protein in Transgenic Plants

### Transmission Electron Microscopy

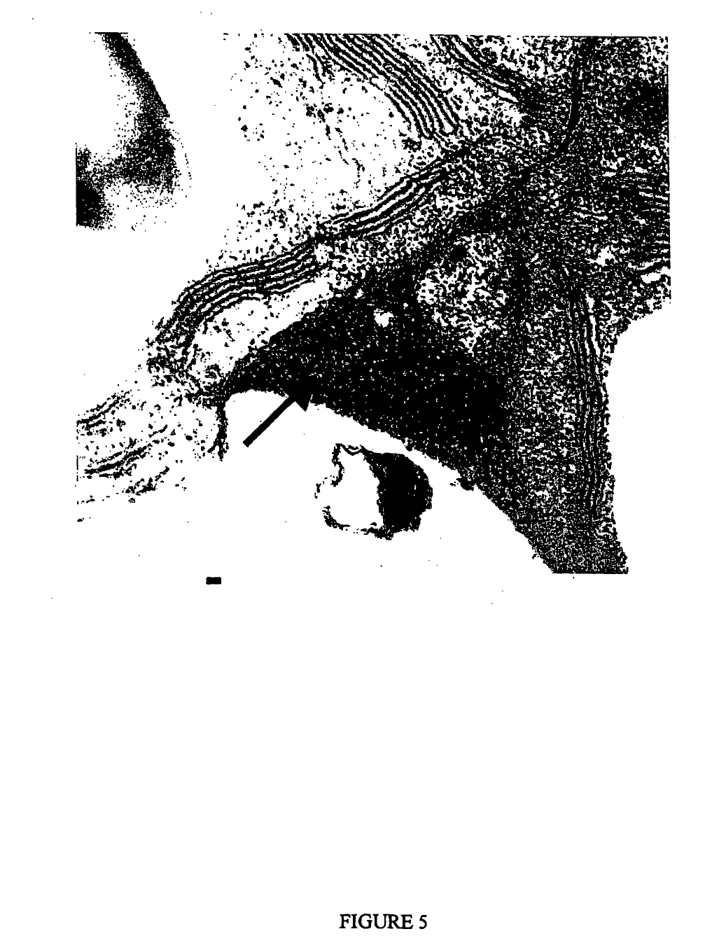

Tissue samples of one transgenic plant, CAVP12-9, which expresses VP2 were analysed by transmission electron microscopy. They were embedded in plastic and sectioned. Structures of approximately 25 nm in diameter, consistent with the size and shape of virus-like particles, were observed in the cytoplasm of the plant cells, as shown in FIG. 5.

### Immunodetection of the VP2 Protein in Plant Extracts

Soluble protein is extracted from 2 g of leaf tissue from each transgenic plant. The tissue is ground to a paste in a chilled mortar and pestle with a small amount of acid-washed sand as an abrasive. 4 mL of extraction buffer comprising 50 mM Tris HCl pH 8.0, 5% glycerol, 0.1 M KCl, 2 mM EDTA, 2 μug/mL, leupeptin, pepstatin and 1.5% polyvinylpolypyrrolidone, is added and the tissue is ground to into a homogenate. The homogenate is filtered through Miracloth™ to remove large particles and then centrifuged at 10,000×g at 4° C. for 20 min. The supernatant is removed, divided into small aliquots and frozen at −20° C. until needed.

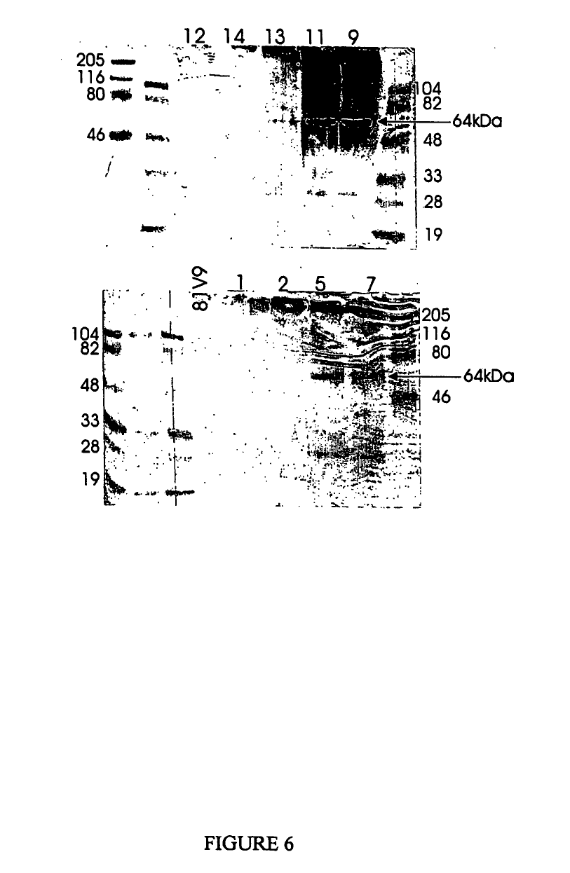

Western blot analysis is performed on the protein extracts using a commercially available monoclonal antibody capable of binding VP2 protein using standard methods. Proteins were separated by SDS-PAGE on a 10% gel, and transferred onto nitrocellulose membrane. The membrane is blocked for 45 minutes at room temperature in TBS containing 10% bovine serum albumin (BSA), then washed once for 5 minutes and then twice for 15 minutes in TBST containing 5% skim milk and incubated with a primary antibody directed against VP2 protein (1/20 dilution). Following an overnight incubation, the membrane is washed, incubated with a secondary antibody (1/1000 dilution) for 45 minutes and then washed 3 times each for 5 minutes in TBST. Following developing of the blot, a protein of approximately 64 kDa cross reacting with the VP2 specific antibody was detected in four of the transgenic plants, as shown in FIG. 6.

### EXAMPLE 5

### Small Animal Trials

### Small Animal Trials by Injection of Protein Composition Comprising VP2

Seed collected from primary transformants was plated on MS media containing 300 mg/mL kanamycin and was allowed to germinate and grow for three weeks. From the germinated seed, green transgenic seedlings were planted in soil-less potting mix and grown for approximately six weeks in a greenhouse. Leaf tissue was then collected and flash frozen in liquid nitrogen, or used fresh. Leaves were coarsely chopped with a razor blade and ground to a pulp with two volumes of buffer (50 mM Tris-HCl, pH 8.0; 0.1 M KCl; 1 mM EDTA; 15 mg/mL PVPP; 5% glycerol) in a pre-chilled Waring blendor. The homogenate was strained through one layer of Miracloth to remove large particles and then centrifuged (8,000×g, 20 min, 4° C.). The supernatant was decanted and centrifuged again using the same conditions. The final supernatant was divided into aliquots and frozen. The same procedure was repeated with untransformed 81V9 plant tissue.

The crude protein extracts were thawed, centrifuged, and filtered through a 0.45 um filter. Groups of 3 to 4 Swiss mice were injected with plant extract. The control group received 125 ug plant extract from untransformed plants per mouse, while the experimental group received 450 ug plant extract from plant CAVP12-9 per mouse. The protein extracts were mixed with equal volumes of Freund's complete adjuvant for the first injection to a total volume of 100 uL, and with Freund's incomplete adjuvant for the second and third injections. Subcutaneous injections were done on days 0, 21 and 42 into the hind legs of the mice, with half of the volume being injected into each leg. Blood was collected before the first injection on day 0, and on day 52.

The serum neutralization assay was done using fetal swine testicle cells (ATCC #CRL 1746). Swine testicle cells were grown for 18 hours at 37° C., 5% CO₂ in MEM in a 48 well tissue culture plate. Mouse serum from the control and experimental mice was heat-inactivated for 30 minutes at 56° C. Serial dilutions of the mouse serum were done in MEM plus two percent fetal bovine serum at 1/300, 1/900, 1/2700, and 1/8100. Porcine parvovirus was diluted to give 50 plaque-forming units/ml. Equal volumes of the diluted PPV and the dilutions of the mouse sera were mixed and incubated for 1.5 hours at 37° C. The PPV plus sera mixtures were then added to the wells of the confluent plate of swine testicle cells (400 uL/well, in duplicate) and incubated at 37° C. for 18 hours. The volume in the wells was increased to 1 ml with MEM and incubation was continued for 4 to 5 days. The number of plaques was counted and the neutralization titre was determined by calculating the dilution which reduced the plaque number by 50 percent.

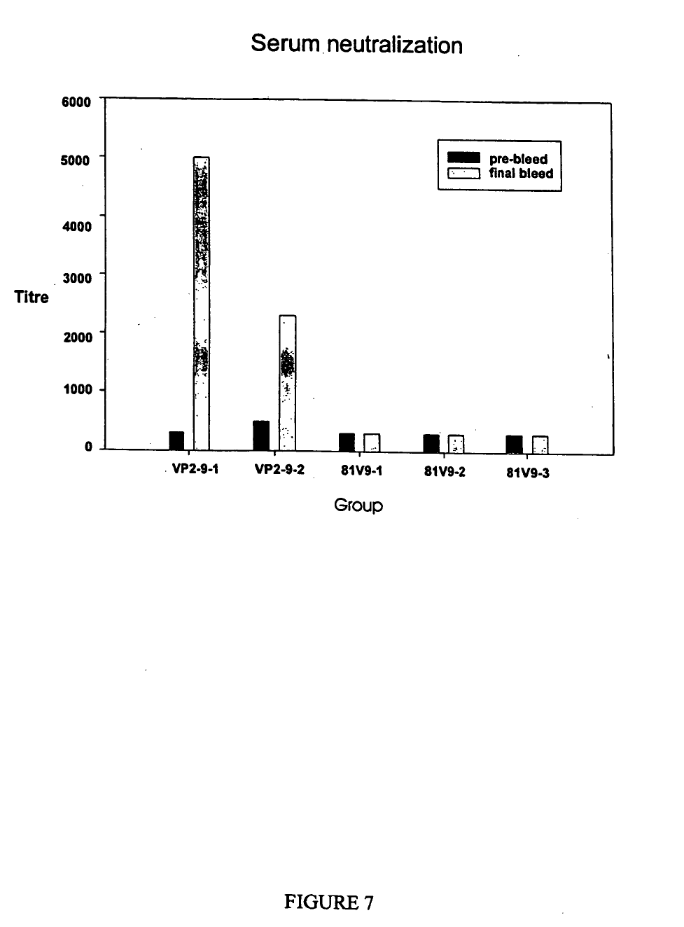

There were no differences in serum neutralization between the pre-bleed and final bleed in the control groups (FIG. 7). However, in the groups injected with plant tissue containing VP2, the serum neutralization titres were considerably higher for the final bleed than for the pre-bleed. These results indicate the production of neutralizing antibodies in mice after immunization with plant produced VP2.

### Small Animal Trials by Oral Feeding

Seed collected from primary transformants is plated on MS media comprising 300 mg/L kanamycin and allowed to germinate and grow for three weeks. From the germinated seed 100 green transgenic seedlings are planted in soil-less potting mix in plug trays and placed in a greenhouse. The plants are allowed to grow for six weeks after which and the leaf material is harvested and flash frozen in liquid nitrogen. This leaf material may be used to prepare pure protein for injection or as a supplement for feeding. Transgenic leaf extracts are prepared by harvesting the leaves and rapidly freezing them in liquid nitrogen. Leaf material is mixed with a small amount of sand and round with a mortar and pestle. After the material is ground to a fine powder, two volumes of buffer (50 mM Tris buffer, 0.1 M KCl, 1 mM EDTA, 5 mM DTT, 15 mg/ml PVPP in 5% glycerol) is added. The larger particles of the homogenate are removed by straining through miracloth and the solution is centrifuged to collect the virus. The pelleted virus is further purified by banding on a sucrose gradient. Following banding on a sucrose gradient, the viral band is emulsified in VSA adjuvant for intramuscular injection or mixed in PBS for oral immunization. In some oral feeding trials, the leaf material may be fed directly to mice as a pelleted chow.

The chow is prepared using transgenic or control leaf material, and is included in a diet at a level of 10% by weight. Assuming VP2 protein concentrations of 5 μg/g in the transgenic leaf material and daily chow intakes of approximately 5 g an oral dose of 25 μg could be expected. The animals are fed with chow containing either transgenic or control leaf material 4 times at days 0, 4, 21 and 25. Following immunization, animals are bled on day 10, 24, and 38 by the tail vein. The levels of antibody are determined by viral neutralization and Western Blots.

### EXAMPLE 6

### Large Animal Trials

Piglets are immunized with different concentrations of purified VLPs by oral delivery (2 μg, 10 μg, or 50 μg/piglet) in saline. Control groups of piglets are immunized intramuscularly with equivalent concentrations of VLPs in VSA adjuvant. The animals are boosted 4 weeks after the primary dosing.

Following immunization, immune responses are monitored by assaying the level of serum antibody present at 2, 4, and 6 weeks post vaccination or boost. Antibody levels are determined using enzyme linked immuno, which are well known in the art (ELISA assays). The level of mucosal immune responses will be determined using an ELISPOT assay or ELISA for the presence of antibody in mucosal secretions.

All references are herein incorporated by reference.

The present invention has been described with regard to preferred embodiments. However, it will be obvious to persons skilled in the art that a number of variations and modifications can be made without departing from the scope of the invention as described herein.

### References

Arakawa, T, Ching, D K X, and Landridge, W H R (1998) Efficacy of food plant based oral cholera toxin B subunit protein. Nature Biotech. 16: 292-297.

Arntzen C J and Lam D M (1999) Proteins expressed in plants (U.S. Pat. No. 5,914,123).

Goodman R M, Knauf V C, Houck C M, Comai L (1997) Molecular Farming (U.S. Pat. No. 5629,175).

Horsch, R. B., Fry, J. E., Hoffman, N. L., Eicholtz, D., Rogers, S. G., and Fraley, R. T. (1985) A simple and general method of transferring genes into plants. Science 227:1229-1231.

Jobling S A, and Gehrke L (1987) Enhanced translation of chimaeric messenger RNAs containing a plant viral untranslated leader sequence. Nature 325:622-625.

Joo H S and Johnson R H (1976) Porcine parvovirus: a review Veterinary Bulletin 46: 653-660.

Kay R, Chan A, Daly M, and McPherson J (1987) Duplication of the 35S promoter sequences creates a strong enhancer for plant genes. Science 236:1299-1302.

Kusnadi, A. R., Nikolov, Z. L. and Howard, J. A. 1997. Production of recombinant proteins in transgenic plants: practical considerations. Biotech Bioeng. 56, 473-484.

Lam D M, Arntzen C J and Mason H S (2000) Proteins expressed in plants (U.S. Pat. No. 6,034,298).

Ma J K C, and Hein M B (1995) Immunotherapeutic potential of antibodies produced in plants TIBTECH 13: 522-527.

Mason H S, Ball J M, Shi J J, Jiang X, Estes M K, and Arntzen C J (1996) Expression of the Norwalk virus capsid protein in transgenic tobacco and its oral immunogenicity in plants. Proc Natl Acad Sci 93:5335-5340.

Martinez, C., Dalsgaard, K., Lopez de Turiso, J. A., Cortes, E., Vela, C., Ingnacio Casal, J. (1992) Production of porcine parvovirus empty capsids with high immunogenic activity. Protein 10:684-690.

Mengeling W L (1978)Prevalence of porcine parvovirus induced reproductive failure: an abortion study. J Amer Vet Res 37: 1393-1399.

Miki B, Hatorri J, Fobert P, Iyer V N (1998). A constitutive promoter from tobacco—U.S. Pat. No. 5824872.

Molitor T W Joo H S, and Collett M S (1984) Porcine parvovirus DNA: characterization of the genomic and replicative form DNA of two virus isolates. Virology 137: 241-254.

Ranz A I, Manclus J J, Diaz-Aroca E and Casal J I (1989) Porcine parvovirus: DNA sequence and genome organization. J Gen Virol 70: 2541-2553.

Woodleif W G, Chaplin J F, Campbell C R, DeJong D W (1981) Effect of variety and harvest treatments on protein yield of close-grown tobacco. Tob Sci 25:83-86.

THE embodiments of the invention in which an exclusive property of privilege is claimed are defined as follows:

1. A method of producing a protein in a plant comprising,
i) transforming said plant with a nucleotide sequence expressing a porcine parvovirus VP2 subunit protein, or a fragment or a derivative thereof, and;
ii) growing said transformed plant.

2. The method of claim 1 further comprising harvesting said plant and administering said plant to an animal.

3 The method of claim 1 further comprising harvesting said plant, purifying said protein and administering said protein to an animal.

4. The method of claim 1 wherein the plant is selected from the group consisting of corn, tobacco, alfalfa, barley and potato.

5. The method of claim 4 wherein the plant is a low nicotine tobacco plant.

6. The method of claim 1 wherein said animal is a pig.

7. The method of claim 1 wherein said animal fetus.

8 The method of claim 1 wherein said nucleotide sequence further comprising at least one regulatory sequence.

9. The method of claim 8 wherein said at least one regulatory sequence comprises promoters, enhancers, transcription termination sequences, polyadenylation sequences, or combinations thereof.

10. The method of claim 1 further wherein said VP2 subunit protein is extracted from said transformed plant and purified or partially purified.

11. The method of claim 10 wherein said protein is further formulated in a suitable excipient for oral, intraperitoneal, subcutaneous, intramuscular or intravenous administration to an animal.

12. A protein composition comprising a porcine parvovirus VP2 subunit protein, fragment or derivative thereof produced by the method of claim 10, and plant extract.

13. The protein composition of claim 12 administered to an animal, said protein capable of conferring immunity against porcine parvovirus in said animal.

14. The method of claim 1 further comprising harvesting of said plant and purifying said protein for the production of a medicament.

15. A use of the protein composition disclosed in claim 12 as a vaccine in an animal.

16. A chimeric construct comprising a regulatory region in operative association with a nucleotide sequence encoding porcine parvovirus VP2 subunit protein, a fragment, or a derivative thereof, said regulatory region being active in a plant.

17. A transgenic plant or progeny produced therefrom comprising the chimeric construct as defined by claim 16.

18. A transgenic plant seed or progeny produced therefrom comprising the chimeric construct as defined by claim 16.

19. A transgenic plant cell or progeny produced therefrom comprising the chimeric construct as defined by claim 16.

20. A protein comprising a porcine parvovirus VP2 subunit protein, fragment or derivative thereof produced by the method of claim 1.

21. The protein of claim 20 administered to an animal, said protein capable of conferring immunity against porcine parvovirus in said animal.


SEQUENCE LISTING (4 sequences)

SEQ ID NO: 1 - DNA, 1802 bp, synthetic VP2subunit protein
ggatcccccctagagtttttatttttaatttttctttcaaatacttccatcatgagtgaaaatgtggaacaacacaaccctattaatgcaggcactgaattgtctgcaacaggaaatgaatctgggggtgggggcggcggtggcgggggtaggggtgctgggggggttggtgtgtctacaggtagtttcaataatcaaacagaatttcaatacttgggggagggcttggttagaatcactgcacacgcatcaagactcatacatctaaatatgccagaacacgaaacatacaaaagaatacatgtactaaattcagaatcaggggtggcgggacaaatggtacaagacgatgcacacacacaaatggtaacaccttggtcactaatagatgctaacgcatggggggtgtggttcaatccagcggactggcagttaatatccaacaacatgacagaaataaacttagttagttttgaacaagaaatattcaatgtagtacttaaaacaattacagaatcagcaacctcaccaccaaccaaaatatataataatgatctaactgcaagcttaatggtcgcactagacaccaataacacacttccatacacaccagcagcacctagaagcgaaacacttggtttttacccatggttacctacaaaaccaactcaatacagatattacctatcatgcaccagaaacctaaatccaccaacatacactggacaatcacaacaaataacagactcaatacaaacaggactacacagtgacattatgttctacacaatagaaaatgcagtaccaattcatcttctaagaacaggagatgaattctccacaggaatatatcactttgacacaaaaccactaaaattaactcactcatggcaaacaaacagatctctaggactgcctccaaaactactaactgaacctaccacagaaggagaccaacacccaggaacactaccagcagctaacacaagaaaaggttatcaccaaacaattaataatagctacacagaagcaacagcaattaggccagctcaggtaggatataatacaccatacatgaattttgaatactccaatggtggaccatttctaactcctatagtaccaacagcagacacacaatataatgatgatgaaccaaatggtgctataagatttacaatgggttaccaacatggacaattaaccacatcttcacaagagctggaaagatacacattcaatccacaaagtaaatgtggaagagctccaaagcaacaatttaatcaacaggcaccactaaacctagaaaatacaaataatggaacacttttaccttcagatccaataggagggaaacctaacatgcatttcatgaatacactcaatacatatggaccattaacagcactaaacaatactgcacctgtatttccaaatggccaaatatgggataaagaacttgatacagatctaaaacctagactacatgttacagctccatttgttcgtaaaaacaatccaccaggacaactatttgtaaaaatagcaccaaacctaacagatgatttcaatgctgactctcctcaacaacctagaataataacttattcaaacttttggtggaaaggaacactaacattcacagcaaaaatgagatccagtaatatgtggaaccctattcaacaacacacaacaacagcagaaaacattggtaactatattcctacaaatattggtggcataaaaatgtttccagaatattcacaacttataccaagaaaattatactagtaataactcta

SEQ ID NO: 2 - DNA, 70 bp, synthetic AMVVP primer
gggtctagagtttttatttttaatttttctttcaaatacttccatcatgagtgaaaatgtggaacaacac

SEQ ID NO: 3 - DNA, 19 bp, synthetic VP1060F
ggtggaccatttctaactc

SEQ ID NO: 4 - DNA, 23 bp, synthetic nos primer
ccggcaacaggattcaatcttaa